(19) United States
(12) Patent Application Publication
(10) Pub. No.: US 2011/0195437 A1
(43) Pub. Date: Aug. 11, 2011

(54) METHOD FOR CHARACTERIZING, IN PARTICULAR FOR QUANTIFYING, MOLECULAR MARKERS THAT ARE INTRACELLULARLY ABSORBED FROM TISSUES BY BLOOD MACROPHAGES THAT ARE RECIRCULATED FROM THE TISSUES INTO THE CIRCULATORY SYSTEM

(75) Inventor: Wolfgang Brozek, Wien (AT)

(73) Assignee: SynMed Research GmbH, Wien (AT)

(21) Appl. No.: 12/737,598

(22) PCT Filed: Aug. 4, 2009
(86) PCT No.: PCT/EP2009/060106

§ 371 Date: Apr. 20, 2011

(30) Foreign Application Priority Data
Aug. 4, 2008 (DE) 10 2008 036 185.2

(51) Int. Cl. G01N 33/53; C12Q 1/02; C12Q 1/34; C12Q 1/28; C12Q 1/42; C12M 1/34

(52) U.S. Cl. 435/7.92; 435/29; 435/18; 435/28; 435/21; 435/287.1

(57) ABSTRACT

The invention relates to a method for characterizing, in particular for quantifying molecular marker(s) that are intracellularly absorbed from tissues by blood macrophages that are recirculated from the tissues into the circulatory system, wherein the following steps are carried out: application of an agent to whole blood, said agent inhibiting coagulation and/or agglomeration of whole blood; selecting and/or enriching and/or separating blood macrophages or leukocyte populations that contain blood macrophages from whole blood; perforating and/or lysing the selected blood macrophages or leukocyte populations containing the blood macrophages, optionally after previous permeabilization thereof; qualitatively and quantitatively determining non blood macrophage markers, namely molecular markers arising from tissue, after previously perforating and/or lysing the blood macrophages or leukocyte populations containing the blood macrophages, and an apparatus for carrying out the method.

The invention relates to a method for characterizing, in particular for quantifying, phagocytosed, intracellular molecular markers, of mononuclear leucocytes of the blood, in particular blood macrophages according to the preamble of claim 1, as well as an analysis assembly for performing the method according to the preamble of claim 13.

In whole blood, cells expressing the proteins both CD14 and CD16 (FcγRIII) on their surface, are detectable. CD14 is a cell surface protein, which is typically expressed by monocytes and macrophages. The cell surface protein CD16 allows binding of the constant (Fc) region of IgG antibodies to cells, it is present in two isoforms: as CD16a (FcγRIIIA) on the surface of NK cells, macrophages and so-called activated monocytes, and as CD16b (FcγRIIIB) on neutrophil granulocytes.

According to the classical doctrine, exclusively macrophages in the tissue and so-called “activated monocytes” of the blood each express both CD14 and CD16. The former doctrine considered CD14/CD16 positive (CD14⁺CD16⁺) cells of the blood simply as “activated monocytes”, which not (yet) have migrated into the tissue, for example at the location of an inflammation, an infection or a tumor (Ziegler-Heitbrock, L., 2007. The CD14⁺CD16⁺ blood monocytes: Their role in infection and inflammation. J. Leukocyte Biol. 81, 584-592).

However, recent findings suggest that activated monocytes are macrophages recirculated from the tissue in the circulatory system, which include tissue portions, e.g. epithelial proteins, in their intracellular phagosomes, which for example originate from the location of an inflammation or of a tumor (Herwig, R., Horninger, W., Rehder, P. et al., 2005. Ability of PSA-positive circulating macrophages to detect prostate cancer. Prostate 62, 290-298; Leers, M. P. G., Nap, M., Herwig, R. et al., 2008. Circulating PSA-containing macrophages as a possible target for the detection of prostate cancer: A three-colour/five-parameter flow cytometric study on peripheral blood samples. Am. J. Clin. Pathol. 129, 649-656). In contrast to the native CD14 positive, CD16 negative (CD14⁺CD16⁻) monocyte population, these cells have the morphological characteristics of well-differentiated macrophages of the tissue, and the (phagocytosed) molecules enclosed by them are not found in monocytes (Leers, M. P. G., Nap, M., Herwig, R. et al., 2008. Circulating PSA-containing macrophages as a possible target for the detection of prostate cancer: A three-colour/five-parameter flow cytometric study on peripheral blood samples. Am. J. Clin. Pathol. 129, 649-656).

Methodically, both for analytically detecting and isolating and enriching CD14⁺CD16⁺ cells of the blood, thus, consideration of the CD16⁺ cells as sub-population of all of the CD14⁺ cells and consideration of the CD14⁺ cells as subset of all of the CD16⁺ cells, is conceivable.

Furthermore, it is known that activated monocytes and blood macrophages, respectively, have a partially weak expression of CD14 (Ziegler-Heitbrock, L., 2007. The CD14⁺CD16⁺ blood monocytes: Their role in infection and inflammation. J. Leukocyte Biol. 81, 584-592) or incur a total loss of CD14 (Bazil and Strominger, 1991: Shedding as a mechanism of down-modulation of CD14 on stimulated human monocytes. J. Immunol. 147, 1567-1574).

The detection of blood macrophages by flow cytometry, which intracellularly contain for example the marker protein PSA (prostate-specific antigen) in their phagosomes, which is then referred to as imPSA (intracellular macrophage PSA), can trace the stadium of diseases of the prostate in extremely specific and sensitive manner, and therein is superior over the classical determination of PSA in the blood serum (Herwig, R., Mitteregger, D., Djavan, B. et al., 2008. Detecting prostate cancer by intracellular macrophage prostate-specific antigen (PSA): A more specific and sensitive marker than conventional serum total PSA. Eur. J. Clin. Invest. 38, 430-437).

The basis of the characterization of cells by flow cytometry is the marking of the target molecules (the “antigens”) to be detected with fluorescent dye labeled antibodies. For analysis, the cells in suspension are passed past a focused laser beam of suitable wavelength by hydrodynamic focusing. Thereby, the fluorescent dye is excited to emit energy in the form of photons, which are registered by a detector.

However, a disadvantage of this approach is in the lacking capability of quantification of the amount of bound antibodies and thus in lacking information on the amount of detected antigen. A further disadvantage is the considerable apparatus and financial expenditure, which the operation of a flow cytometer causes.

The object of the invention is in specifying an improved, simplified and inexpensive method as well as an analysis assembly, with the aid of which characterization of mononuclear leucocytes of the blood, in particular blood macrophages, including the phagocytosed molecular markers thereof, from tissue is quantitatively possible, in particular by representation as mass unit per cell count.

According to the invention, this object is solved by a method according to claim 1 as well as by an apparatus according to claim 13.

In particular, the object is solved by a method for characterizing, in particular quantifying, molecular marker(s) that are intracellularly absorbed from tissue by blood macrophages that are recirculated from the tissue into the circulatory system, wherein the following steps are performed:
applying an agent to whole blood, which inhibits coagulation and/or agglomeration of whole blood;
performing a selection and/or enrichment and/or separation of blood macrophages or leukocyte populations containing blood macrophages from the whole blood;
performing a permeabilization and/or perforation and/or lysis of the selected blood macrophages or leukocyte populations containing blood macrophages;
performing a qualitative and quantitative determination of non blood macrophage markers, namely from molecular markers arising from tissue, after previous perforation and/or lysis of the blood macrophages or leukocyte populations containing blood macrophages.

An inventively principal consideration is in detecting molecular markers, e.g. epithelial proteins, in phagocytosed mononuclear cells of the circulatory system, namely in blood macrophages, wherein based on the method according to the invention it is advantageously possible to provide a direct and specific as well as quantitative detection.

In order to allow detection of the molecular markers in blood macrophages, therefore, it is provided according to the invention to first perform a selection and/or enrichment and/or separation of blood macrophages from whole blood, wherein afterwards the terms “activated monocyte” and “macrophage” are to be understood as identical to “blood macrophage”. Further, it is pointed out that all of the markers and marker fragments treated within the scope of this invention and to be detected, respectively, are at least partially phagocytosed molecules absorbed by macrophages in the tissue, which are intracellularly detected in blood macrophages.

According to a preferred embodiment of the invention, the selection and/or enrichment and/or separation of the blood macrophages is performed by means of a positive selection using antibodies directed against the surface marker CD16.

Thus, according to the invention, consideration of all CD16 expressing cells is performed in order to ensure detection of all activated monocytes/macrophages.

If desired, according to the invention, it is further provided that either preferably after or according to an alternative also before the positive selection using antibodies directed against the surface marker CD16, a positive selection using antibodies directed against the surface marker CD14 is performed. The previous performance of positive CD16⁺ selection using antibodies directed against the surface marker CD16 before positive CD14⁺ selection offers the advantage according to the invention that the CD14⁺ population of undifferentiated monocytes, which is large with respect to the CD16⁺ population, which do not have CD16 surface markers and which are not subject matter of the method according to the invention for characterizing, in particular quantifying, molecular marker(s) that are intracellularly absorbed from tissue by blood macrophages that are recirculated from the tissue into the circulatory system, is already excluded in a first step.

With this approach, it is advantageously ensured that only CD14⁺CD16⁺ cells are selected, wherein it is mentioned that positive CD 16⁺ selection after positive CD14⁺ selection is also possible according to the invention.

If other cell populations with the cell surface protein CD16, in particular NK cells, should be viewed as disturbing in the following analysis of the molecular markers contained in intracellular phagosomes and arising from tissue, a negative selection based on the surface proteins CD56 or CD57 or CD161 expressed exclusively by NK cells under CD16⁺ mononuclear cells can be done afterwards.

For this reason, according to the invention, a negative selection for excluding cells with the surface markers CD56 and/or CD57 and/or CD161 using antibodies can be advantageously performed, which are directed against the surface markers CD56 and/or CD57 and/or CD161. Such a negative selection can be performed as a separate selection step or, according to a further advantageous embodiment, else simultaneously with a positive selection of CD16⁺ cells, as is exemplarily explained below.

Furthermore, according to the invention, it is provided that a positive or negative selection is performed using magnetic beads, which can be, preferably reversibly, coupled, in order to separate the respectively selected cells by means of a suitable magnet in extremely simple manner.

Alternatively or in preceding or succeeding selection steps, according to the invention, it is proposed to perform a positive or negative selection using antibodies coupled to an ELISA plate, in order to increase the sample throughput on the one hand, and to avoid unnecessary transfers of the liquid to be examined from a vessel into another one and substance loss of the cells to be examined associated therewith on the other hand in inventively advantageous manner.

Further, according to a further preferred embodiment according to the invention, a quantitative determination of a mononuclear leukocyte population in an Elisa plate, preferably by measuring the lactate dehydrogenase activity by means of an Elisa plate reader, particularly preferred in the same Elisa plate, in which a qualitative and quantitative determination of the molecular markers, in particular non blood macrophage antigens, is performed, is provided.

Furthermore, the performance of a quantitative determination of the present amount of non blood macrophage molecular markers arising from tissue by means of Elisa plate reader and/or chemiluminescence measuring device is performed, wherein, according to the invention, as molecular markers, in particular tissue markers and/or serological markers, abnormal DNA methylation, AFP (α-1-fetoprotein), AHCY (S-adenosyl-homocysteine-hydrolase), AMY2 (pancreas amylase), CA 15-3 (synonyms: MUC1, EMA, CD227), CA 19-9, CA 50, CA 72-4 synonym: TAG72), CA 125 (synonym: MUC16), calcitonin, calprotectin, CCSA-2 (colon cancer-specific antigen-2), CCSP-2 (colon cancer secreted protein-2), CEA to (carcinoembryonic antigen), CYP24A1, cytokeratin (CK) 8 and fragments thereof, CK18 and fragments thereof, CK19 and fragments thereof, CRP (C-reactive protein), cystatin B, DDH (dihydrodiol-dehydrogenase), DKK-1 (Dikkopf-1), GP73 (Golgi protein-73) (synonym: GOLPH2), HE4 (human epididymis protein 4), HER2/neu, HSP (heat shock protein)-27, Mac-2 BP (Mac-2 binding protein), mammaglobin A, mammaglobin B, MIA (melanoma-inhibitory activity), MnSOD (manganese superoxide dismutase), PARK7 (synonym: DJ-1), ProGRP (progastrin-releasing peptide), NSE (neuron-specific enolase), pan-cytokeratin, Pro-MMP (pro-matrix-metalloproteinase)-7, PSA (prostate-specific antigen), S100A8, S100A9, S-100beta, SCCA1 (squamous cell carcinoma antigen 1), SCCA2 (squamous cell carcinoma antigen 2), thyroglobulin, UHRF1 (ubiquitin-like with/containing PHD and ring-finger domains 1), URG4 (up-regulated gene 4), and YKL-40 (synonym: CHI3-L1), also in combination(s), are used.

According to the invention, for the purpose of performing the method, whole blood is first mixed with an agent against coagulation and/or agglomeration, for example heparin or another suitable anticoagulant, e.g. citrate solution.

A perforation or lysis of the macrophages or leukocyte populations containing macrophages is further performed according to the invention for example by means of a saponin treatment or a treatment with triton solution.

Furthermore, according to the invention, for determining non blood macrophage antigens, there are used antibodies optionally conjugated with biotin, HRP (horseradish peroxidase), AP (alkaline phosphatase) or luminescent dyes, in particular of the immunoglobulin class G (IgG) and Fab or F(ab)₂ fragments, and/or aptamers, in particular selected from: anti-mouse IgG (polyclonal), anti-rabbit IgG (polyclonal), anti-goat IgG (polyclonal), anti-rat IgG (polyclonal), anti-donkey IgG (polyclonal), anti-AFP (clone AFP-01), anti-AFP (clone AFP-11), anti-AFP (clone 4A3), anti-AFP (clone 5H7), anti-AFP (clone M803209), anti-AFP (clone M0151611), anti-AFP (clone M0151608), anti-AFP (polyclonal), anti-AHCY (clone 1E11-1A7), anti-AHCY (clone 2F11-1D10), anti-AHCY (clone 4H2), anti-AHCY (clone M1), anti-AHCY (clone M2), anti-AHCY (polyclonal), anti-AMY2 (clone 6A9/1), anti-AMY2 (clone 501), anti-AMY2 (clone 503), anti-AMY2 (clone 10-102.5), anti-AMY2 (polyclonal), anti-CA 15-3/MUC1 (clone M2C5), anti-CA 15-3/MUC1 (clone M9E7), anti-CA 15-3/MUC1 (clone M4H2), anti-CA 15-3/MUC1 (clone M8C9), anti-CA 15-3/MUC1 (clone M10G4), anti-CA 15-3/MUC1 (clone M1OH6), anti-CA 15-3/MUC1 (clone M3A106), anti-CA 15-3/MUC1 (clone C595(NCRC48)), anti-CA 15-3/MUC1 (clone E29), anti-CA 15-3/MUC1 (polyclonal), anti-CA 19-9 (clone 121SLE), anti-CA 19-9 (polyclonal), anti-CA 50 (clone M991149), anti-CA 50 (clone 93), anti-CA 50 (polyclonal), anti-CA 72-4/TAG72 (clone SPM148), anti-CA 72-4/TAG72 (polyclonal), anti-CA 125/MUC16 (clone 2F1), anti-CA 125/MUC16 (clone 10G12), anti-CA 125/MUC16 (clone X75), anti-CA 125/MUC16 (clone X325), anti-CA 125/MUC16 (polyclonal), anti-calcitonin (clone SP17), anti-calcitonin (clone 13B9), anti-calcitonin (clone 13f2), anti-calcitonin (clone 24B2), anti-calcitonin (polyclonal), anti-calprotectin (clone 27E10), anti-calprotectin (polyclonal), anti-CCSA-2 (polyclonal), anti-CCSP-2 (polyclonal), anti-CEA (clone Col-1), anti-CEA (clone 1C7), anti-CEA (clone 1C10), anti-CEA (clone 1C11), anti-CEA (polyclonal), anti-CYP24A1 (clone 1E1), anti-CYP24A1 (clone 1F8), anti-CYP24A1 (polyclonal), anti-CK8 (clone 24), anti-CK8 (clone LP3K), anti-CK8 (polyclonal), anti-CK18 (clone DC-10), anti-CK18 (clone DA-7), anti-CK18 (clone LDK18), anti-CK18 (polyclonal), anti-CK19 (clone A53-B/A2), anti-CK19 (clone BA17), anti-CK19 (clone 236-11221), anti-CK19 (polyclonal), anti-CRP (clone 232007), anti-CRP (clone 232024), anti-CRP (clone C2), anti-CRP (clone C4), anti-CRP (clone C5), anti-CRP (clone C6), anti-CRP (clone C7), anti-CRP (polyclonal), anti-cystatin B (clone 2F1), anti-cystatin B (clone 8k275), anti-cystatin B (clone B-02), anti-cystatin B (clone RJMW-2E7), anti-cystatin B (polyclonal), anti-DDH (clone T101), anti-DDH (polyclonal), anti-DKK-1 (clone 141135), anti-DKK-1 (polyclonal), anti-GP73/GOLPH2 (clone YA-14), anti-GP73/GOLPH2 (clone 5B10), anti-GP73/GOLPH2 (polyclonal), anti-HE4 (clone C-12), anti-HE4 (polyclonal), anti-HER2/neu (clone 1007), anti-HER2/neu (clone 191924), anti-HER2/neu (clone N3/D10), anti-HER2/neu (polyclonal), anti-HSP-27 (clone G3.1), anti-HSP-27 (clone AF5E5), anti-HSP-27 (clone F-4), anti-HSP-27 (clone 2A5), anti-HSP-27 (polyclonal), anti-Mac-2 BP (clone SP-2), anti-Mac-2 BP (polyclonal), anti-mammaglobin A (clone 1G8D6, synonym 2E7G9), anti-mammaglobin A (clone 304-1A5), anti-mammaglobin A (polyclonal), anti-mammaglobin B (clone E-17), anti-mammaglobin B (polyclonal), anti-MIA (clone 3A6), anti-MIA (polyclonal), anti-MMP-7 (clone 6A4), anti-MMP-7 (clone 176-5F12), anti-MMP-7 (clone 141-7B2), anti-MMP-7 (clone 377313), anti-MMP-7 (polyclonal), anti-MnSOD (clone 1AE), anti-MnSOD (clone 2A1), anti-MnSOD (clone 4F10), anti-MnSOD (clone 23G5), anti-MnSOD (clone 37CT127.5.11.6), anti-MnSOD (polyclonal), anti-PARK7/DJ-1 (clone 1B11), anti-PARK7/DJ-1 (clone 1D7), anti-PARK7/DJ-1 (clone 6A65), anti-PARK7/DJ-1 (clone 3055), anti-PARK7/DJ-1 (clone A-9), anti-PARK7/DJ-1 (clone D-4), anti-PARK7/DJ-1 (clone E2), anti-PARK7/DJ-1 (polyclonal), anti-proGRP (clone pGRP5), anti-proGRP (clone E146), anti-proGRP (clone E172), anti-GRP (clone 76-E6), anti-proGRP (polyclonal), anti-GRP (polyclonal), anti-NSE (clone 1C1), anti-NSE (clone 5A4), anti-NSE (clone 5E2), anti-NSE (clone 5G10), anti-NSE (polyclonal), anti-pan-cytokeratin (clone 7H8C4), anti-pan-cytokeratin (clone B311.1), anti-pan-cytokeratin (clone C11), anti-pan-cytokeratin (clone D-12), anti-pan-cytokeratin (polyclonal), anti-PSA (clone ER-PR8), anti-PSA (clone 181823), anti-PSA (polyclonal), anti-S100A8 (clone 1B3), anti-S100A8 (clone 2C5/4), anti-S100A8 (clone 2H2), anti-S100A8 (clone 2Q396A), anti-S100A8 (clone 6A614), anti-S100A8 (clone 8L627), anti-S100A8 (clone 8-5C2), anti-S100A8 (clone CF-145), anti-S100A8 (clone MRP8 7C12/4), anti-S100A8 (clone S13.67), anti-S 100A8 (polyclonal), anti-S 100A9 (clone 1 C 10), anti-S 100A9 (clone 2Q396B), anti-S100A9 (clone 4G9), anti-S100A9 (clone NO.19), anti-S100A9 (clone NO.134), anti-S100A9 (clone S32.2), anti-S100A9 (clone S36.48), anti-S100A9 (polyclonal), anti-S-100beta (clone SB6), anti-S-100beta (clone SH-B1), anti-S-100beta (clone SH-B4), anti-S-100beta (polyclonal), anti-SCCA1 (clone 8H11), anti-SCCA1 (polyclonal), anti-SCCA2 (clone 10C12), anti-SCCA2 (polyclonal), anti-SCCA1/2 (clone B-9), anti-SCCA1/2 (polyclonal), anti-thyroglobulin (clone 5E6), anti-thyroglobulin (clone 5F9), anti-thyroglobulin (clone 5G4), anti-thyroglobulin (clone 11A16), anti-thyroglobulin (clone PB2), anti-thyroglobulin (clone PB3), anti-thyroglobulin (polyclonal), anti-UHRF1 (clone 1RC1C-10), anti-UHRF1 (clone 3A11), anti-UHRF1 (polyclonal), anti-URG4 (polyclonal), anti-YKL-40/CHI3-L1 (clone 2011), anti-YKL-40/CHI3-L1 (clone 321806), anti-YKL-40/CHI3-L1 (polyclonal).

Thus, in summary, it can be stated that first all of the mononuclear cells are extracted by density gradient centrifugation, thereby excluding CD16 positive neutrophil granulocytes. A positive selection and enrichment of either cells with the surface protein CD14 or cells with the surface protein CD16 succeeds thereto. As an alternative, a negative selection of cells with the surface protein CD56 or CD57 or CD161 can be effected instead.

According to the invention, both the positive selection and enrichment of mononuclear cells with the surface proteins CD14 and CD16 and the negative selection of mononuclear cells with the surface proteins CD56 or CD57 or CD161 are effected with the aid of antibodies coupled to magnetic beads, which are to directed against the cell surface proteins CD14 or CD16 or CD56 or CD57 or CD161.

In case of a positive selection against CD14⁺ cells or negative selection, a further separation step characterized by positive selection of the cells using an antibody directed against the cell surface protein CD16, which is either coupled to magnetic beads or coated to an Elisa plate, follows. In case of a positive selection against first CD16⁺ cells, a further positive selection of the cells against CD14⁺ cells with the aid of an antibody, which is either coupled to magnetic beads or coated to an Elisa plate, follows, or a negative selection against CD56⁺ or CD57⁺ or CD161⁺ NK cells using a magnetic antibody directed against the cell surface protein CD56 or CD57 or CD161 follows. As an alternative, positive selection against CD16⁺ cells and negative selection against CD56⁺ or CD57⁺ or CD161⁺ NK cells can be performed in one method step at the same time. In case of a positive selection against CD16⁺ cells, alternatively thereto, further selection steps can be omitted.

In further succession, the now separated and enriched mononuclear leukocytes are destroyed by treatment with a perforation solution and/or a cell lysis reagent. According to a method variant, beforehand, either immediately before the lysis of the cells or immediately after the density gradient centrifugation, the antibody/antibodies can be added to the molecular marker(s) to be detected with permeabilization of the cells. Thus, in one of the steps following the cell lysis, the quantification of the molecular markers contained in the cells is possible.

Furthermore, for the purpose of determining the inserted cell count, the activity of the enzyme lactate dehydrogenase (LDH) in the lysate is determined in an Elisa plate, wherein the amount of present mononuclear leukocytes can be calculated via a standard. By adding an indicator substrate, the color reaction induced by the enzymatic activity can be read by means of Elisa reader and thus be quantified.

Lastly, by means of antibody/antibodies specifically directed against the respective molecular marker and/or by means of antibody/antibodies directed against the respective molecular antibody/antibodies, preferably in the same Elisa plate, in which the cell quantification has been effected by measuring the LDH activity, an enzymatic color reaction or a chemiluminescence signal is induced proportionally to the amount of the examined marker. By means of Elisa plate reader or chemiluminescence detector, the respective molecular marker can be quantified via a standard. The final representation of the results is effected as the “amount or mass unit on intracellular molecular marker per cell count”.

The object according to the invention is further solved by an analysis assembly for performing the method according to the invention according to claim 13.

According to the invention, the analysis assembly for performing the method includes
a device for adding an agent inhibiting coagulation and/or agglomeration of whole blood (e.g. heparin, citrate solution or another suitable anticoagulant);
a device for performing a pre-selection or selection and enrichment of all of the mononuclear leukocytes present in the blood sample by means of density gradient centrifugation;
a device for performing a selection and enrichment of CD14⁺ cells using antibodies against CD14, which are, optionally reversibly, coupled to magnetic beads or to an Elisa plate;
a device for performing a selection and enrichment of CD16⁺ cells using antibodies against CD16, which are, optionally reversibly, coupled to magnetic beads or to an Elisa plate;
optionally, a device for excluding NK cells with the cell surface markers CD56 or CD57 or CD161 from the collective of mononuclear leukocytes or mononuclear CD16⁺ cells using antibodies against CD56 or CD57 or CD161, which are coupled to magnetic beads;
a device for perforation and/or lysis of the mononuclear leukocyte populations;
a device for quantitative determination of the mononuclear leukocyte populations in an Elisa plate (e.g. measurement of the lactate dehydrogenase activity by means of Elisa plate reader), in the same Elisa plate, in which the qualitative and quantitative determination of the molecular markers takes place;
a device for qualitative and quantitative determination of intracellular molecular markers phagocytosed by blood macrophages (e.g. with the aid of Elisa plate reader, chemiluminescence measuring device etc.) after preceding perforation of lysis of the blood cells.

Further, according to the invention, a device for fixation and permeabilization of mononuclear leukocytes can be provided before lysis thereof, preferably before or optionally after the selection and/or enrichment and/or separation of the blood macrophages or leukocyte populations containing blood macrophages, in order to advantageously perform an intracellular binding of antibodies directed against intracellularly phagocytosed molecular markers to be quantified already before the cell lysis.

Below, the invention is explained in more detail by way of several embodiments:

### 1^{st} EMBODIMENT

Positive selection of the CD14⁺ monocytes with the aid of magnetic beads;

positive selection of the CD14⁺CD16⁺ population, cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken and stored at 2-8° C. The suspension is mixed with an adequate amount of magnetic beads, which bear an antibody directed against the cell surface antigen CD14 directly on their surface, for example 10⁸ beads in 250 μl. Alternatively thereto, the suspension can be incubated with 10 μg of an antibody conjugated with modified DSB-X biotin against the cell surface antigen CD14 at 2-8 ° C. for 10 minutes with shaking or swinging, whereupon the suspension is centrifuged (350×g, 8 minutes), after discarding the supernatant, the cells are resuspended in 0.5 ml PBS solution (pH 7.4) containing 0.1% BSA and 2 mM EDTA, but not any Ca²⁺ or Mg²⁺ ions. An adequate amount of magnetic beads bearing streptavidin on their surface, for example 10⁸ beads in 300 μl, is added.

After the suspension of mononuclear leukocytes and magnetic beads is shaken or swung for 20 minutes at 2-8° C., the cells now bound to the magnetic beads are separated with the aid of a suitable magnet, wherein the supernatant is discarded. The beads are washed several times with a PBS solution (pH 7.4) containing 0.1% BSA. Upon use of magnetic beads bearing an antibody directed against the cell surface antigen CD14 on their surface, the beads are suspended in 0.5 ml PBS solution (pH 7.4) containing 0.1% BSA and 2 mM EDTA, but not any Ca²⁺ or Mg²⁺ ions. Alternatively thereto, upon use of the antibody conjugated with modified DSB-X biotin against the cell surface antigen CD14 and the magnetic beads bearing streptavidin on their surface, the beads can be resuspended in a cell release buffer containing modified biotin. The suspension is shaken or swung for 10 minutes at room temperature, followed by the magnetic separation of beads and supernatant. The supernatant is removed, centrifuged (350×g, 8 minutes) and suspended in 0.5 ml PBS solution (pH 7.4) containing 0.1% BSA and 2 mM EDTA, but not any Ca²⁺ or Mg²⁺ ions.

A defined volume of the homogenized suspension (maximum 100 μl) is pipetted per well into a 96 well Maxi-Sorp plate, which is coated with both a mouse antibody directed against the cell surface antigen CD16, for example 5 μg/ml of the clone 3G8, and with a mouse antibody directed against PSA, for example 1 μg/ml of the clone ER-PR8, and has been blocked (for example with a 5 FCS (fetal calf serum) solution). Dilution series of the suspension can be produced. An aliquot of the suspension is to be kept at 2-8° C. The plate is sealed (for example with cling film or aluminum foil) and incubated for one hour at room temperature or over night in the refrigerator at 2-8° C.

The plate is washed five times with PBS solution (pH 7.4) containing 0.1% BSA and 2 mM EDTA, but not any Ca²⁺ or Mg²⁺ ions. Subsequently, 50 μl cell lysis solution containing 1% triton and 1 mM PMSF (phenyl-methyl-sulfonyl-fluoride) are pipetted into each well. In addition, aliquots of the kept suspensions of all mononuclear leukocytes or of the beads with all cells bearing the antigen CD14 on their surface are pipetted in any number of, for example respectively two, wells and mixed with the volume of cell lysis solution deficient to 50 μl, which contains 1% triton and 1 mM PMSF (phenyl-methyl-sulfonyl-fluoride). Furthermore, a dilution series of recombinant PSA, for example between 50 μg/ml and 2 ng/ml, is prepared as a standard, of which 50 μl of each concentration are pipetted into each at least two free wells. Now, the plate is set to 2-8° C. for 5 minutes and subsequently treated in the ultrasonic bath for 5 minutes (only the plate bottom immerses in the water bath, a cage mounted slightly below the water surface serves as a support).

After sealing the plate and incubating at room temperature (one hour) or at 2-8° C. (over night), a dilution series of the calibrated LDH positive control in cell lysis solution, for example starting from a 1/2500 dilution, is prepared as a standard, of which 50 μl of each concentration are pipetted into each at least two free wells. Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 gg/ml in PBS, for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD14⁺CD16⁺ cells or else CD14⁺ cells or else mononuclear leukocytes).

### 2^{nd} EMBODIMENT

Positive selection of the CD16⁺ monocytes with the aid of magnetic beads; positive selection of the CD14⁺CD16⁺ population, cell count determination, and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken and stored at 2-8° C. The suspension is mixed with an adequate amount of magnetic beads, which bear an antibody directed against the cell surface antigen CD16 directly on their surface, for example 10⁸ beads in 250 μl. Alternatively thereto, the suspension can be incubated with 10 μg of an antibody conjugated with modified DSB-X biotin against the cell surface antigen CD16 at 2-8° C. for 10 minutes with shaking or swinging, whereupon the suspension is centrifuged (350×g, 8 minutes), after discarding the supernatant, the cells are resuspended in 0.5 ml PBS solution (pH 7.4) containing 0.1% BSA and 2 mM EDTA, but not any Ca²⁺ or Mg²⁺ ions. An adequate amount of magnetic beads bearing streptavidin on their surface, for example 10⁸ beads in 300 μl, is added.

After the suspension of mononuclear leukocytes and magnetic beads is shaken or swung for 20 minutes at 2-8° C., the cells now bound to the magnetic beads are separated with the aid of a suitable magnet, wherein the supernatant is discarded. The beads are washed several times with a PBS solution (pH 7.4) containing 0.1% BSA. Upon use of magnetic beads bearing an antibody directed against the cell surface antigen CD16 on their surface, the beads are suspended in 0.5 ml PBS solution (pH 7.4) containing 0.1% BSA and 2 mM EDTA, but not any Ca²⁺ or Mg²⁺ ions. Alternatively thereto, upon use of the antibody conjugated with modified DSB-X biotin against the cell surface antigen CD16 and the magnetic beads bearing streptavidin on their surface, the beads can be resuspended in a cell release buffer containing modified biotin. The suspension is shaken or swung for 10 minutes at room temperature, followed by the magnetic separation of beads and supernatant. The supernatant is removed, centrifuged (350×g, 8 minutes) and suspended in 0.5 ml PBS solution (pH 7.4) containing 0.1% BSA and 2 mM EDTA, but not any Ca²⁺ or Mg²⁺ ions.

A defined volume of the homogenized suspension (maximum 100 μl) is pipetted per well into a 96 well Maxi-Sorp plate, which is coated with both a mouse antibody directed against the cell surface antigen CD14, for example 5 μg/ml of the clone MφP9, and with a mouse antibody directed against PSA, for example 1 μg/ml of the clone ER-PR8, and has been blocked (for example with a 5% FCS (fetal calf serum) solution). Dilution series of the suspension can be produced. An aliquot of the suspension is to be kept at 2-8° C. The plate is sealed (for example with cling film or aluminum foil) and incubated for one hour at room temperature or over night in the refrigerator at 2-8° C.

The plate is washed five times with PBS solution (pH 7.4) containing 0.1% BSA and 2 mM EDTA, but not any Ca²⁺ or Mg²⁺ ions. Subsequently, 50 μl cell lysis solution containing 1% triton and 1 mM PMSF (phenyl-methyl-sulfonyl-fluoride) are pipetted into each well. In addition, aliquots of the kept suspensions of all mononuclear leukocytes or of the beads with all cells bearing the antigen CD16 on their surface are pipetted in any number of, for example respectively two, wells and mixed with the volume of cell lysis solution deficient to 50 μl, which contains 1% triton and 1 mM PMSF (phenyl-methyl-sulfonyl-fluoride). Furthermore, a dilution series of recombinant PSA, for example between 50 μg/ml and 2 ng/ml, is prepared as a standard, of which 50 μl of each concentration are pipetted into each at least two free wells. Now, the plate is set to 2-8° C. for 5 minutes and subsequently treated in the ultrasonic bath for 5 minutes (only the plate bottom immerses in the water bath, a cage mounted slightly below the water surface serves as a support).

After sealing the plate and incubating at room temperature (one hour) or at 2-8° C. (over night), a dilution series of the calibrated LDH positive control in cell lysis solution, for example starting from a 1/2500 dilution, is prepared as a standard, of which 50 μl of each concentration are pipetted into each at least two free wells. Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS, for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD14⁺CD16⁺ cells or else CD16⁺ cells or else mononuclear leukocytes).

### 3^{rd} EMBODIMENT

Positive selection of both first the CD14⁺ monocytes and subsequently the CD14⁺CD16⁺ population with the aid of magnetic beads; cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken, dissolved in a defined volume of cell lysis solution containing 1% triton and 1 mM PMSF (phenyl-methyl-sulfonyl-fluoride), and stored at 2-8° C. The suspension is incubated with 10 μg of an antibody conjugated with modified DSB-X biotin against the cell surface antigen CD14 at 2-8° C. for 10 minutes with shaking or swinging.

The cells are centrifuged (350×g, 8 minutes) and, after discarding the supernatant, resuspended in 0.5 ml PBS solution (pH 7.4) containing 0.1% BSA and 2 mM EDTA, but not any Ca²⁺ or Mg²⁺ ions. An adequate amount of magnetic beads bearing streptavidin on their surface, for example 10⁸ beads in 300 μl, is added.

Thereafter, the suspension of mononuclear leukocytes and magnetic beads is shaken or swung for 20 minutes at 2-8° C. Therein, the beads labeled with streptavidin bind to antibody conjugated with DSB-X biotin, which are bound to cells with the surface antigen CD14.

The separation of the cells bound to the magnetic beads with the aid of a suitable magnet follows thereto, wherein the supernatant is discarded. The beads are washed several times with a PBS solution (pH 7.4) containing 0.1% BSA and 2 mM EDTA, but not any Ca²⁺ or Mg²⁺ ions, and subsequently resuspended in a cell release buffer containing modified biotin. The suspension is shaken or swung for 10 minutes at room temperature, followed by the magnetic separation of beads and supernatant. The supernatant is removed, centrifuged (350×g, 8 minutes) and suspended in 0.5 ml PBS solution (pH 7.4) containing 0.1% BSA and 2 mM EDTA, but not any Ca²⁺ or Mg²⁺ ions. An aliquot of the suspension is removed is and centrifuged, the pellet is dissolved in a defined volume of cell lysis solution containing 1% triton and 1 mM PMSF (phenyl-methyl-sulfonyl-fluoride) and kept at 2-8° C.

Now, the cell suspension is mixed with an adequate amount of magnetic beads bearing an antibody directed against the cell surface antigen CD16 on their surface, for example 4×10⁷ beads in 100 μl, and shaken or swung for 20 minutes at 2-8° C. The separation of the cells bound to the magnetic beads with the aid of a suitable magnet follows, wherein the supernatant is discarded. The beads with the cells are washed several times with a PBS solution (pH 7.4) containing 0.1% BSA and 2 mM EDTA, but not any Ca²⁺ or Mg²⁺ ions, before a defined volume of cell lysis solution containing 1% triton and 1 mM PMSF (phenyl-methyl-sulfonyl-fluoride) is added. The lysate is set to 2-8° C. for at least 5 minutes.

Now, all of the cell lysates are treated in the ultrasonic bath for 5 minutes, a suitable magnet is used for removing the beads from the corresponding lysates, and they are once again centrifuged (14000×g, 10 minutes). A defined volume of the supernatants (maximum 100 μl) is pipetted per well into a 96 well Maxi-Sorp plate, which is coated with a mouse antibody directed against PSA, for example 1 μg/ml of the clone ER-PR8, and has been blocked (for example with a 5% FCS (fetal calf serum) solution). A dilution series of recombinant PSA, for example between 50 pg/ml and 2 ng/ml, is prepared as a PSA standard, of which a defined volume of each concentration is pipetted into each at least two free wells.

Furthermore, a dilution series of the calibrated LDH positive control in cell lysis solution, for example starting from a 1/2500 dilution, is prepared as a cell count standard, of which 50 μl of each concentration are pipetted into each at least two free wells. The plate is sealed (for example with cling film or aluminum foil) and incubated for one hour at room temperature.

Subsequently, 50 μl LDH substrate solution are added to each incubated well of the plate, the plate is sealed impermeable to light with aluminum foil and left for 30 minutes at room temperature. Thereafter, all of the incubated wells are mixed to with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the cell count standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS solution (pH 7.4), for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD14⁺CD16⁺ cells or else CD14⁺ cells or else mononuclear leukocytes).

### 4^{th} EMBODIMENT

Positive selection of both first the CD16⁺ population and subsequently the CD14⁺CD16⁺ population with the aid of magnetic beads; cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken, dissolved in a defined volume of cell lysis solution containing 1% triton and 1 mM PMSF (phenyl-methyl-sulfonyl-fluoride), and stored at 2-8° C. The suspension is incubated with 5 μg of an antibody conjugated with modified DSB-X biotin against the cell surface antigen CD16 at 2-8° C. for 10 minutes with shaking or swinging.

The cells are centrifuged (350×g, 8 minutes) and, after discarding the supernatant, resuspended in 0.5 ml PBS solution (pH 7.4) containing 0.1% BSA and 2 mM EDTA, but not any Ca²⁺ or Mg²⁺ ions. An adequate amount of magnetic beads bearing streptavidin on their surface, for example 2×10⁷ beads in 50 μl, is added.

Thereafter, the suspension of mononuclear leukocytes and magnetic beads is shaken or swung for 20 minutes at 2-8° C. Therein, the beads labeled with streptavidin bind to antibody conjugated with DSB-X biotin, which are bound to cells with the surface antigen CD16.

The separation of the cells bound to the magnetic beads with the aid of a suitable magnet follows thereto, wherein the supernatant is discarded. The beads are washed several times with a PBS solution (pH 7.4) containing 0.1% BSA and 2 mM EDTA, but not any Ca²⁺ or Mg²⁺ ions, and subsequently resuspended in a cell release buffer containing modified biotin. The suspension is shaken or swung for 10 minutes at room temperature, followed by the magnetic separation of beads and supernatant. The supernatant is removed, centrifuged (350×g, 8 minutes) and suspended in 0.5 ml PBS solution (pH 7.4) containing 0.1% BSA and 2 mM EDTA, but not any Ca²⁺ or Mg²⁺ ions. A defined volume of the suspension is removed from it and centrifuged, the pellet is dissolved in a defined volume of cell lysis solution containing 1% triton and 1 mM PMSF (phenyl-methyl-sulfonyl-fluoride) and kept at 2-8° C.

Now, the cell suspension is mixed with an adequate amount of magnetic beads bearing an antibody directed against the cell surface antigen CD14 on their surface, for example 2×10⁷ beads in 50 μl, and shaken or swung for 20 minutes at 2-8° C. The separation of the cells bound to the magnetic beads with the aid of a suitable magnet follows, wherein the supernatant is discarded. The beads with the cells are washed several times with a PBS solution (pH 7.4) containing 0.1% BSA and 2 mM EDTA, but not any Ca²⁺ or Mg²⁺ ions, before a defined volume of cell lysis solution containing 1% triton and 1 mM PMSF (phenyl-methyl-sulfonyl-fluoride) is added. The lysate is set to 2-8° C. for at least 5 minutes.

Now, all of the cell lysates are treated in the ultrasonic bath for 5 minutes, a suitable magnet is used for removing the beads from the corresponding lysates, and they are once again centrifuged (14000×g, 10 minutes). A defined volume of the supernatants (maximum 100 μl) is pipetted per well into a 96 well Maxi-Sorp plate, which is coated with a mouse antibody directed against PSA, for example 1 μg/ml of the clone ER-PR8, and has been blocked (for example with a 5% FCS (fetal calf serum) solution). A dilution series of recombinant PSA, for example between 50 pg/ml and 2 ng/ml, is prepared as a PSA standard, of which a defined volume of each concentration is pipetted into each at least two free wells. Furthermore, a dilution series of the calibrated LDH positive control in cell lysis solution, for example starting from a 1/2500 dilution, is prepared as a cell count standard, of which 50 μl of each concentration are pipetted into each at least two free wells. The plate is sealed (for example with cling film or aluminum foil) and incubated for one hour at room temperature.

Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the cell count standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS solution (pH 7.4), for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD14⁺CD16⁺ cells or else CD16⁺ cells or else mononuclear leukocytes).

### 5^{th} EMBODIMENT

Positive selection of the CD16⁺ population with the aid of magnetic beads; cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken, dissolved in a defined volume of cell lysis solution containing 1% triton and 1 mM PMSF (phenyl-methyl-sulfonyl-fluoride), and stored at 2-8° C. The suspension is mixed with an adequate amount of magnetic beads bearing an antibody directed against the cell surface antigen CD16 on their surface, for example 2×10⁷ beads in 50 μl. As an alternative, 5 μg of an antibody conjugated with modified DSB-X biotin against the cell surface antigen CD16 can be incubated at 2-8° C. for 10 minutes with shaking or swinging, whereupon the addition of an adequate amount of magnetic beads bearing streptavidin on their surface, for example 2×10⁷ beads in 50 μl, follows.

Thereafter, the suspension of mononuclear leukocytes and magnetic beads is shaken or swung for 20 minutes at 2-8° C. Therein, the beads coupled to antibody or the beads labeled with streptavidin bind to cells bearing the antigen CD16 on their surface or to antibody conjugated with DSB-X biotin, which are bound to cells with the surface antigen CD16.

The separation of the cells bound to the magnetic beads with the aid of a suitable magnet follows thereto, wherein the supernatant is discarded. The beads are washed several times with a PBS solution (pH 7.4) containing 0.1% BSA and 2 mM EDTA, but not any Ca²⁺ or Mg²⁺ ions, and subsequently dissolved in a defined volume of cell lysis solution containing 1% triton and 1 mM PMSF (phenyl-methyl-sulfonyl-fluoride) and kept at 2-8° C.

Now, all of the cell lysates are treated in the ultrasonic bath for 5 minutes, a suitable magnet is used for removing the beads from the corresponding lysates, and they are once again centrifuged (14000×g, 10 minutes). A defined volume of the supernatants (maximum 100 μl) is pipetted per well into a 96 well Maxi-Sorp plate, which is coated with a mouse antibody directed against PSA, for example 1 μg/ml of the clone ER-PR8, and has been blocked (for example with a 5% FCS (fetal calf serum) solution). A dilution series of recombinant PSA, for example between 50 μg/ml and 2 ng/ml, is prepared as a PSA standard, of which a defined volume of each concentration is pipetted into each at least two free wells. Furthermore, a dilution series of the calibrated LDH positive control in cell lysis solution, for example starting from a 1/2500 dilution, is prepared as a cell count standard, of which 50 μl of each concentration are pipetted into each at least two free wells. The plate is sealed (for example with cling film or aluminum foil) and incubated for one hour at room temperature.

Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the cell count standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS solution (pH 7.4), for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at to 570 nm is subtracted. The intensity of the measured absorption is proportional to the amount of imPSA, which can be determined based on the standard.

The representation of the results is for example effected as pg imPSA/cell count (CD16⁺ cells or else mononuclear leukocytes).

### 6^{th} EMBODIMENT

Positive selection of the CD16⁺ population, subsequently negative selection of the CD16⁺CD56⁻ or CD16⁺CD57⁻or CD16⁺CD161− population with the aid of magnetic beads; cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken, dissolved in a defined volume of cell lysis solution containing 1% triton and 1 mM PMSF (phenyl-methyl-sulfonyl-fluoride), and stored at 2-8° C. The suspension is mixed with an adequate amount of magnetic beads bearing an antibody directed against the cell surface antigen CD16 on their surface, for example 2×10⁷ beads in 50 μl. As an alternative, 5 μg of an antibody conjugated with modified DSB-X biotin against the cell surface antigen CD16 can be incubated at 2-8° C. for 10 minutes with shaking or swinging, whereupon the addition of an adequate amount of magnetic beads bearing streptavidin on their s surface, for example 2×10⁷ beads in 50 μl, follows.

Thereafter, the suspension of mononuclear leukocytes and magnetic beads is shaken or swung for 20 minutes at 2-8° C. Therein, the beads coupled to antibody or the beads labeled with streptavidin bind to cells bearing the antigen CD16 on to their surface or to antibody conjugated with DSB-X biotin, which are bound to cells with the surface antigen CD16.

The separation of the cells bound to the magnetic beads with the aid of a suitable magnet follows thereto, wherein the supernatant is discarded. The beads are washed several times with a PBS solution (pH 7.4) containing 0.1% BSA and 2 mM EDTA, but not any Ca²⁺ or Mg²⁺ ions, and subsequently resuspended in a cell release buffer containing modified biotin. The suspension is shaken or swung for 10 minutes at room temperature followed by the magnetic separation of beads and supernatant. The supernatant is removed, centrifuged (350×g, 8 minutes) and suspended in 0.5 ml PBS solution (pH 7.4) containing 0.1% BSA and 2 mM EDTA, but not any Ca²⁺ or Mg²⁺ ions. A defined volume of the suspension is removed from it and centrifuged, the pellet is dissolved in a defined volume of cell lysis solution containing 1% triton and 1 mM PMSF (phenyl-methyl-sulfonyl-fluoride) and kept at 2-8° C.

Now, the cell suspension is mixed with an adequate amount of magnetic beads bearing an antibody directed against the cell surface antigen CD56 or CD57 or CD161 on their surface, for example 2×10⁷ beads in 50 μl. As an alternative, 5 μg of an antibody conjugated with modified DSB-X biotin against the cell surface antigen CD56 or CD57 or CD161 can be incubated at 2-8° C. for 10 minutes with shaking or swinging, whereupon the addition of an adequate amount of magnetic beads bearing streptavidin on their surface, for example 2×10⁷ beads in 50 μl, follows.

After 20 minutes of shaking or swinging at 2-8° C., the separation of the cells bound to the magnetic beads with the aid of a suitable magnet follows, wherein the supernatant is kept. The beads with the cells are washed several times with a PBS solution (pH 7.4) containing 0.1% BSA and 2 mM EDTA, but not any Ca²⁺ or Mg²⁺ ions, the washing solution is added to the supernatant and also kept, the beads are discarded. The supernatant solution is centrifuged (350×g, 8 minutes), a defined volume of cell lysis solution containing 1% triton and 1 mM PMSF (phenyl-methyl-sulfonyl-fluoride) is added to the cell pellet. The lysate is set to 2-8° C. for at least 5 minutes.

Now, all of the cell lysates are treated in the ultrasonic bath for 5 minutes and once again centrifuged (14000×g, 10 minutes). A defined volume of the supernatants (maximum 100 μl) is pipetted per well into a 96 well Maxi-Sorp plate, which is coated with a mouse antibody directed against PSA, for example 1 μg/ml of the clone ER-PR8, and has been blocked (for example with a 5% FCS (fetal calf serum) solution). A dilution series of recombinant PSA, for example between 50 pg/ml and 2 ng/ml, is prepared as a PSA standard, of which a defined volume of each concentration is pipetted into each at least two free wells. Furthermore, a dilution series of the calibrated LDH positive control in cell lysis solution, for example starting from a 1/2500 dilution, is prepared as a cell count standard, of which 50 μl of each concentration are pipetted into each at least two free wells. The plate is sealed (for example with cling film or aluminum foil) and incubated for one hour at room temperature.

Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the cell count standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS solution (pH 7.4), for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD16⁺CD56⁻ or CD16⁺CD57⁻ or CD16⁺CD16⁺ cells or else CD16⁺ cells or else mononuclear leukocytes).

### 7^{th} EMBODIMENT

Negative selection of the CD56⁻ or CD57⁻ or CD161⁻ population, subsequently positive selection of the CD16⁺CD56⁻ or CD16⁺CD57⁻ or CD16⁺CD161⁻ population with the aid of magnetic beads; cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken, dissolved in a defined volume of cell lysis solution containing 1% triton and 1 mM PMSF (phenyl-methyl-sulfonyl-fluoride), and stored at 2-8° C. The suspension is mixed with an adequate amount of magnetic beads bearing an antibody directed against the cell surface antigen CD56 or CD57 or CD161 on their surface, for example 2×10⁷ beads in 50 μl. As an alternative, 5 μg of an antibody conjugated with modified DSB-X biotin against the cell surface antigen CD56 or CD57 or CD161 can be incubated at 2-8° C. for 10 minutes with shaking or swinging, whereupon the addition of an adequate amount of magnetic beads bearing streptavidin on their surface, for example 2×10⁷ beads in 50 μl, follows.

Thereafter, the suspension of mononuclear leukocytes and magnetic beads is shaken or swung for 20 minutes at 2-8° C. Therein, the beads coupled to antibody or the beads labeled with streptavidin bind to cells bearing the antigen CD56 or CD57 or CD161 on their surface or to antibody conjugated with DSB-X biotin, which are bound to cells with the surface antigen CD56 or CD57 or CD161.

The separation of the cells bound to the magnetic beads with the aid of a suitable magnet follows thereto, wherein the supernatant is kept. The beads are washed several times with a PBS solution (pH 7.4) containing 0.1% BSA and 2 mM EDTA, but not any Ca²⁺ or Mg²⁺ ions, the washing solution is added to the supernatant and also kept, the beads are discarded. The supernatant solution is centrifuged (350×g, 8 minutes), the pellet is suspended in 0.5 ml PBS solution (pH 7.4) containing 0.1% BSA and 2 mM EDTA, but not any Ca²⁺ or Mg²⁺ ions. A defined volume of the suspension is removed from it and centrifuged, the pellet is dissolved in a defined volume of cell lysis solution containing 1% triton and 1 mM PMSF (phenyl-methyl-sulfonyl-fluoride) and kept at 2-8° C.

Now, the cell suspension is mixed with an adequate amount of magnetic beads bearing an antibody directed against the cell surface antigen CD16 on their surface, for example 2×10⁷ beads in 50 μl. As an alternative, 5 μg of an antibody conjugated with modified DSB-X biotin against the cell surface antigen CD16 can be incubated at 2-8° C. for 10 minutes with shaking or swinging, whereupon the addition of an adequate amount of magnetic beads bearing streptavidin on their surface, for example 2×10⁷ beads in 50 μl, follows.

After 20 minutes of shaking or swinging at 2-8° C., the separation of the cells bound to the magnetic beads with the aid of a suitable magnet follows, wherein the supernatant is discarded. The beads with the cells are washed several times with a PBS solution (pH 7.4) containing 0.1% BSA and 2 mM EDTA, but not any Ca²⁺ or Mg²⁺ ions, and subsequently dissolved in a defined volume of cell lysis solution containing 1% triton and 1 mM PMSF (phenyl-methyl-sulfonyl-fluoride) and set to 2-8° C. for at least 5 minutes.

Now, all of the cell lysates are treated in the ultrasonic bath for 5 minutes and once again centrifuged (14000×g, 10 minutes). A defined volume of the supernatants (maximum 100 μl) is pipetted per well into a 96 well Maxi-Sorp plate, which is coated with a mouse antibody directed against PSA, for example 1 μg/ml of the clone ER-PR8, and has been blocked (for example with a 5% FCS (fetal calf serum) solution). A dilution series of recombinant PSA, for example between 50 pg/ml and 2 ng/ml, is prepared as a PSA standard, of which a defined volume of each concentration is pipetted into each at least two free wells. Furthermore, a dilution series of the calibrated LDH positive control in cell lysis solution, for example starting from a 1/2500 dilution, is prepared as a cell count standard, of which 50 μl of each concentration are pipetted into each at least two free wells. The plate is sealed (for example with cling film or aluminum foil) and incubated for one hour at room temperature.

Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the cell count standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS solution (pH 7.4), for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD16⁺CD56⁻ or CD16⁺CD57⁻ or CD16⁺CD161⁻ cells or else CD56⁻ or CD57⁻ or CD161⁻ cells or else mononuclear leukocytes).

### 8^{th} EMBODIMENT

Negative selection of the CD56⁻ or CD57⁻ or CD161⁻ population with the aid of magnetic beads; positive selection of the CD16⁺CD56⁻ or CD16⁺CD57⁻ or CD16⁺CD161⁻ population; cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken, dissolved in a defined volume of cell lysis solution containing 1% triton and 1 mM PMSF (phenyl-methyl-sulfonyl-fluoride), and stored at 2-8° C. The suspension is mixed with an adequate amount of magnetic beads bearing an antibody directed against the cell surface antigen CD56 or CD57 or CD161 on their surface, for example 2×10⁷ beads in 50 μl. As an alternative, 5 μg of an antibody conjugated with modified DSB-X biotin against the cell surface antigen CD56 or CD57 or CD 161 can be incubated at 2-8° C. for 10 minutes with shaking or swinging, whereupon the addition of an adequate amount of magnetic beads bearing streptavidin on their surface, for example 2×10⁷ beads in 50 μl, follows.

Thereafter, the suspension of mononuclear leukocytes and magnetic beads is shaken or swung for 20 minutes at 2-8° C. Therein, the beads coupled to antibody or the beads labeled with streptavidin bind to cells bearing the antigen CD56 or CD57 or CD161 on their surface or to antibody conjugated with DSB-X biotin, which are bound to cells with the surface antigen CD56 or CD57 or CD161.

The separation of the cells bound to the magnetic beads with the aid of a suitable magnet follows thereto, wherein the supernatant is kept. The beads are washed several times with a PBS solution (pH 7.4) containing 0.1% BSA and 2 mM EDTA, but not any Ca²⁺ or Mg²⁺ ions, the washing solution is added to the supernatant and also kept, the beads are discarded. The supernatant solution is centrifuged (350×g, 8 minutes), the pellet is suspended in 0.5 ml PBS solution (pH 7.4) containing 0.1% BSA and 2 mM EDTA, but not any Ca²⁺ or Mg²⁺ ions.

A defined volume of the cell suspension (maximum 100 μl) is pipetted per well into a 96 well Maxi-Sorp plate, which is coated with both a mouse antibody directed against the cell surface antigen CD16, for example 5 μg/ml of the clone 3G8, and with a mouse antibody directed against PSA, for example 1 μg/ml of the clone ER-PR8, and has been blocked (for example with a 5 FCS (fetal calf serum) solution). Dilution series of the suspension can be prepared. A residual volume of the suspension is to be kept, but not frozen at the moment. The plate is sealed (for example with cling film or aluminum foil) and incubated for one hour at room temperature or over night in the refrigerator at 2-8° C.

The plate is washed five times with PBS solution (pH 7.4) containing 0.1% BSA and 2 mM EDTA, but not any Ca²⁺ or Mg²⁺ ions. Subsequently, 50 μl cell lysis solution containing 1% triton and 1 mM PMSF (phenyl-methyl-sulfonyl-fluoride) are pipetted into each well. In addition, 25 μl of the kept suspension of all cells not bearing the antigen CD56 or CD57 or CD161 on their surface are pipetted in any number of, for example two, wells and mixed with the same volume of cell lysis solution, which contains 1% triton and 1 mM PMSF (phenyl-methyl-sulfonyl-fluoride). Furthermore, a dilution series of recombinant PSA, for example between 50 pg/ml and 2 ng/ml, is prepared as a standard, of which 50 μl of each concentration are pipetted into each at least two free wells. Now, the plate is set to 2-8° C. for 5 minutes and subsequently treated in the ultrasonic bath for 5 minutes (only the plate bottom immerses in the water bath, a cage mounted slightly below the water surface serves as a support).

After sealing the plate and incubating at room temperature (one hour) or at 2-8° C. (over night), a dilution series of the calibrated LDH positive control in cell lysis solution, for example starting from a 1/2500 dilution, is prepared as a standard, of which 50 μl of each concentration are pipetted into each at least two free wells. Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS solution (pH 7.4), for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD16⁺CD56⁻ or CD16⁺CD57⁻ or CD16⁺CD161⁻ cells or else CD56⁻ or CD57⁻ or CD161⁻ cells or else mononuclear leukocytes).

### 9^{th} EMBODIMENT

Positive selection of the CD16⁺CD56⁻ or CD16⁺CD57⁻ or CD16⁺CD161⁻ population with the aid of one-time application of magnetic beads; cell count determination and quantification of the molecular marker, here imPSA, in an Elisa plate.

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, an aliquot can be taken, dissolved in a defined volume of cell lysis solution containing 1% triton and 1 mM PMSF (phenyl-methyl-sulfonyl-fluoride), and stored at 2-8° C. The suspension is mixed with 5 μg of an antibody conjugated with modified DSB-X biotin against the cell surface antigen CD16 and with the same amount (5 μg) of an antibody conjugated with biotin against the cell surface antigen CD56 or CD57 or CD161. The suspension is incubated at 2-8° C. for 10 minutes with shaking or swinging, whereupon the addition of an adequate amount of magnetic beads bearing streptavidin on their surface, for example 2×10⁷ beads in 50 μl, follows.

Thereafter, the suspension of mononuclear leukocytes and magnetic beads is shaken or swung for 20 minutes at 2-8° C. Therein, the beads labeled with streptavidin bind to antibodies conjugated with biotin as well as with DSB-X biotin, which are bound to cells with the surface antigen CD56 or CD57 or CD161 or with the surface antigen CD 16.

The separation of the cells bound to the magnetic beads with the aid of a suitable magnet follows thereto, wherein the supernatant is discarded. The beads are to washed several times with a PBS solution (pH 7.4) containing 0.1% BSA and 2 mM EDTA, but not any Ca²⁺ or Mg²⁺ ions, the washing solution is discarded, the beads are subsequently resuspended in a cell release buffer containing modified biotin. The suspension is shaken or swung for 10 minutes at room temperature, followed by the magnetic separation of beads and supernatant. The supernatant only containing CD16⁺CD56⁻ or CD16⁺CD57⁻ or CD16⁺CD161⁻ cells is removed, centrifuged (350×g, 8 minutes), the pellet is dissolved in a defined volume of cell lysis solution containing 1% triton and 1 mM PMSF (phenyl-methyl-sulfonyl-fluoride) and set to 2-8° C. for at least 5 minutes.

Now, all of the cell lysates are treated in the ultrasonic bath for 5 minutes and once again centrifuged (14000×g, 10 minutes). A defined volume of the supernatants (maximum 100 μl) is pipetted per well into a 96 well Maxi-Sorp plate, which is coated with a mouse antibody directed against PSA, for example 1 μg/ml of the clone ER-PR8, and has been blocked (for example with a 5 FCS (fetal calf serum) solution). A dilution series of recombinant PSA, for example between 50 pg/ml and 2 ng/ml, is prepared as a PSA standard, of which a defined volume of each concentration is pipetted into each at least two free wells. Furthermore, a dilution series of the calibrated LDH positive control in cell lysis solution, for example starting from a 1/2500 dilution, is prepared as a cell count standard, of which 50 μl of each concentration are pipetted into each at least two free wells. The plate is sealed (for example with cling film or aluminum foil) and incubated for one hour at room temperature.

Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the cell count standard.

Now, all of the wells are washed five times with PBS solution containing 0.05% or 0.1% Tween-20. Thereupon, the wells are incubated with a polyclonal detection antibody of goat directed against PSA, for example in a concentration of 0.5 μg/ml in PBS solution (pH 7.4), for one hour at room temperature.

All of the wells are now again washed five times with PBS solution containing 0.05% or 0.1% Tween-20. A secondary antibody, for example of mouse or rabbit, directed against the detection antibody and conjugated with the enzyme HRP (horseradish peroxidase), for example in a concentration of 2 μg/ml in PBS solution (pH 7.4), is added. The incubation time at room temperature is again one hour.

After all of the wells have been again washed five times with PBS solution containing 0.05% or 0.1% Tween-20, 100 μl of a substrate solution containing TMB (tetramethyl-benzidine) are added per well. The incubation time is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD16⁺CD56⁻ or CD16⁺CD57⁻ or CD16⁺CD161⁻ cells or else mononuclear leukocytes).

### 10^{th} EMBODIMENT

Isolation of the CD14⁺CD16⁺ or CD16⁺ or CD16⁺CD56⁻ or CD16⁺CD57⁻ or CD16⁺CD161⁻ population with the aid of magnetic beads, as described in the 3^{rd}, 4^{th}, 5^{th}, 6^{th}, 7^{th} and 9^{th} embodiment; either beforehand or thereafter, addition of an antibody against the molecular marker, here imPSA, with permeabilization of the cells; cell count determination and quantification of the molecular marker, here imPSA, in an Elisa plate.

Performance described for isolation of the CD16⁺CD56⁻ or CD16⁺CD57⁻ or CD16⁺CD161⁻ population according to the 9^{th} embodiment:

It is started with 6 ml whole blood, which is taken in a tube containing a polyester gel and a 0.1 M sodium citrate solution. By centrifugation of the blood (for example 1800×g, 20 minutes), all of the mononuclear leukocytes are isolated together with the thrombocytes above the gel.

The isolated cells are washed twice with PBS (phosphate buffered saline) solution (pH 7.4), which additionally can contain 0.1% BSA (bovine serum albumin) and 2 mM EDTA (ethylene-diamine-tetra-acetate) in order to remove the thrombocytes.

The isolated mononuclear leukocytes are suspended in 0.5 ml PBS solution (pH 7.4), which contains 0.1% BSA and 2 mM EDTA, but not any Ca²⁺ or Mg²⁺ ions, and mixed with 5 μg of an antibody conjugated with modified DSB-X biotin against the cell surface antigen CD16 and with the same amount (5 μg) of an antibody conjugated with biotin against the cell surface antigen CD56 or CD57 or CD161. The suspension is incubated at 2-8° C. for 10 minutes with shaking or swinging. After a washing step with PBS solution (pH 7.4), the cells are suspended in 0.5 ml fixing reagent (for example 1% formalin solution), washed with PBS solution (pH 7.4) after 10 min, thereafter received in 100 μl permeabilization medium and incubated with a mouse antibody directed against

PSA and conjugated with the enzyme HRP (horseradish peroxidase) or with biotin, for example 1 μg/ml of the clone ER-PR8, for ca. 20 minutes. Thereafter, the cells are washed with a PBS solution (pH 7.4) and suspended in 0.5 ml PBS solution (pH 7.4) containing 0.1% BSA and 2 mM EDTA, but not any Ca²⁺ or Mg²⁺ ions; an aliquot can be taken, dissolved in a defined volume of cell lysis solution containing 1% triton and 1 mM PMSF (phenyl-methyl-sulfonyl-fluoride), and stored at 2-8° C. The addition of an adequate amount of magnetic beads bearing streptavidin on their surface, for example 2×10⁷ beads in 50 μl, follows thereto. Alternatively thereto, the treatment of the cells with fixing reagent and permeabilization medium can be omitted at this point of the protocol, wherein after removal of an aliquot, which is dissolved in a defined volume of cell lysis solution containing 1% triton and 1 mM PMSF (phenyl-methyl-sulfonyl-fluoride) and stored at 2-8° C., the cells are immediately incubated with an adequate amount of magnetic beads bearing streptavidin on their surface, for example 2×10⁷ beads in 50 μl.

Thereafter, the suspension of mononuclear leukocytes and magnetic beads is shaken or swung for 20 minutes at 2-8° C. Therein, the beads labeled with streptavidin bind to antibodies conjugated with biotin as well as with DSB-X biotin, which are bound to cells with the surface antigen CD56 or CD57 or CD161 or with the surface antigen CD16.

The separation of the cells bound to the magnetic beads with the aid of a suitable magnet follows thereto, wherein the supernatant is discarded. The beads are washed several times with a PBS solution (pH 7.4) containing 0.1% BSA and 2 mM EDTA, but not any Ca²⁺ or Mg²⁺ ions, the washing solution is discarded, the beads are subsequently resuspended in a cell release buffer containing modified biotin. The suspension is shaken or swung for 10 minutes at room temperature, followed by the magnetic separation of beads and supernatant. The supernatant only containing CD16⁺CD56⁻ or CD16⁺CD57⁻ or CD16⁺CD161⁻ cells is removed and centrifuged (350×g, 8 minutes). After discarding the supernatant, if addition of the antibody directed against imPSA with permeabilization of the cells already occurred, the pellet is dissolved in a defined volume of cell lysis solution containing 1% triton and 1 mM PMSF (phenyl-methyl-sulfonyl-fluoride) and set to 2-8° C. for at least 5 minutes. Else, the pellet is suspended in 0.5 ml fixing reagent (for example 1% formalin suspension) after discarding the supernatant, washed with PBS solution (pH 7.4) after 10 min, thereafter received in 100 μl permeabilization medium and incubated with a mouse antibody directed against PSA and conjugated with the enzyme HRP (horseradish peroxidase) or biotin, for example 1 μg/ml of the clone ER-PR8, for ca. 20 minutes. Thereafter, the cells are washed with a PBS solution (pH 7.4) and dissolved in a defined volume of cell lysis solution containing 1% triton and 1 mM PMSF (phenyl-methyl-sulfonyl-fluoride) and set to 2-8° C. for at least 5 minutes.

Now, all of the cell lysates are treated in the ultrasonic bath for 5 minutes and once again centrifuged (14000×g, 10 minutes). A defined volume of the supernatants (maximum 100 μl) is pipetted per well into a 96 well Maxi-Sorp plate, which is coated with a polyclonal antibody directed against mouse immunoglobulin (in case of use of an antibody conjugated with HRP and directed against PSA), for example in a concentration of 2 μg/ml, or with avidin or streptavidin (in case of use of an antibody conjugated with biotin and directed against PSA) and has been blocked (for example with a 5% FCS (fetal calf serum) solution). A dilution series of the mouse antibody clone ER-PR8 conjugated with the enzyme HRP (horseradish peroxidase) or with biotin is prepared as a standard, of which a defined volume of each concentration is pipetted into each at least two free wells. From this, it can be calculated back to the amount of bound imPSA. Furthermore, a dilution series of the calibrated LDH positive control in cell lysis solution, for example starting from a 1/2500 dilution, is prepared as a cell count to standard, of which 50 μl of each concentration are pipetted into each at least two free wells. The plate is sealed (for example with cling film or aluminum foil) and incubated for one hour at room temperature.

Subsequently, 50 μl LDH substrate solution are added to each incubated well of the plate, the plate is sealed impermeable to light with aluminum foil and left for 30 minutes at room temperature. Thereafter, all of the incubated wells are mixed with each 50 μl stopping solution (1M CH₃COOH), all of the large air bubbles are punctured with a needle, and the light absorptions of all wells are measured in an Elisa plate reader at 492 nm. The intensity of the measured absorption is proportional to the cell count, which can be determined based on the cell count standard.

Now, all of the wells are washed five times with PBS solution containing 0.05% or 0.1% Tween-20, and mixed with 100 μl of a substrate solution containing TMB (tetramethyl-benzidine) in case of use of an antibody conjugated with HRP and directed against PSA. In case of use of an antibody conjugated with biotin and directed against PSA, beforehand, it is incubated with a polyclonal antibody conjugated with HRP and directed against mouse immunoglobulin, for example in a concentration of 0.5 μg/ml, for one hour, thereafter, all of the wells are washed five times with PBS solution containing 0.05% or 0.1% Tween-20. The incubation time with substrate solution containing TMB is 15 minutes at room temperature, thereafter, 50 μl of a stopping solution (for example 1 M H₂SO₄) are pipetted into each well. Finally, the light absorption of the wells is measured in an Elisa plate reader at a wavelength of 450 nm, the absorption at 570 nm is subtracted. The intensity of the measured absorption is proportional to the amount of imPSA, which can be determined based on the standard.

The representation of the results is for example effected as pg imPSA/cell count (CD16⁺CD56⁻ or CD16⁺CD57⁻ or CD16⁺CD161⁻ cells or else mononuclear leukocytes).

At this point, it is to be mentioned that all of the above mentioned embodiments, which exemplarily demonstrate the quantification of imPSA, can explicitly also be applied to characterization of all of the other molecular markers mentioned above, in particular those mentioned in claim 9, and the quantification of these markers is effected in analog manner to the quantification of imPSA. Furthermore, it is to be pointed out that for determining the respective molecular markers or the respective epitopes thereof, the antibodies respectively associated with these markers and/or epitopes are used.

At this point, it is to be pointed out that all of the above described parts in themselves and in each combination, in particular the details represented in the drawing, are claimed as essential for the invention. The man skilled in the art is familiar with modifications hereto.

1. A method for characterizing, in particular quantifying, molecular marker(s) that are intracellularly absorbed from tissue by blood macrophages that are recirculated from the tissue into the circulatory system,
characterized by:
the following steps:
applying an agent to whole blood, which inhibits coagulation and/or agglomeration of whole blood;
performing a selection and/or enrichment and/or separation of blood macrophages or leukocyte populations containing blood macrophages from the whole blood;
performing a perforation and/or lysis of the selected blood macrophages or leukocyte populations containing blood macrophages, optionally after preceding optional permeabilization of the selected blood macrophages or leukocyte populations containing blood macrophages;
performing a qualitative and quantitative determination of phagocytosed, non blood macrophage markers, namely from molecular markers arising from tissue, after previous perforation and/or lysis of the blood macrophages or leukocyte populations containing blood macrophages.

2. The method according to claim 1,
characterized in that
the selection and/or enrichment and/or separation of the blood macrophages are performed by means of a positive selection using antibodies directed against the surface marker CD16.

3. The method according to claim 2,
characterized in that
preferably after or optionally before the positive selection using antibodies directed against the surface marker CD16, a positive selection using antibodies directed against the surface marker CD14 is performed.

4. The method according to claim 1,
characterized in that
a negative selection for excluding cells with the surface markers CD56 and/or CD57 and/or CD161 is performed using antibodies directed against the surface markers CD56 and/or CD57 and/or CD161.

5. The method according to claim 1,
characterized in that
a positive or negative selection is performed using magnetic beads, which can be, preferably reversibly, coupled.

6. The method according to claim 1,
characterized in that
a positive or negative selection is performed using antibodies coupled to an ELISA plate.

7. The method according to claim 1,
characterized in that
a quantitative determination of the mononuclear leukocyte populations is performed in an Elisa plate, preferably by measurement of the lactate dehydrogenase activity by means of Elisa plate reader, particularly preferred in the same Elisa plate, in which a qualitative and quantitative determination of the molecular markers, in particular non blood macrophage antigens.

8. The method according to claim 1,
characterized in that
the performance of a quantitative determination of the present amount of phagocytosed, non blood macrophage molecular markers arising from tissue is performed by means of Elisa plate reader and/or chemiluminescence measuring device.

9. The method according to claim 1,
characterized in that
as the molecular markers, in particular tissue markers and/or serologic markers, there are used
abnormal DNA methylation;
AFP (α-1-fetoprotein);
AHCY (S-adenosyl-homocysteine-hydrolase);
AMY2 (pancreas-amylase);
CA 15-3 (synonyms: MUC1, EMA, CD227);
CA 19-9;
CA 50;
CA 72-4 (synonym: TAG72);
CA 125 (synonym: MUC16);
calcitonin;
calprotectin;
CCSA-2 (colon cancer-specific antigen-2);
CCSP-2 (colon cancer secreted protein-2);
CEA (carcinoembryonic antigen);
CYP24A1;
cytokeratin (CK) 8 and fragments thereof;
CK18 and fragments thereof;
CK19 and fragments thereof;
CRP (C-reactive protein);
cystatin B;
DDH (dihydrodiol-dehydrogenase);
DKK-1 (Dikkopf-1);
GP73 (Golgi protein-73) (synonym: GOLPH2);
HE4 (human epididymis protein 4);
HER2/neu;
HSP (heat shock protein)-27;
Mac-2 BP (Mac-2 binding protein);
mammaglobin A;
mammaglobin B;
MIA (melanoma-inhibitory activity);
MnSOD (manganese-superoxide dismutase);
PARK7 (synonym: DJ-1);
ProGRP (progastrin-releasing peptide);
NSE (neuron-specific enolase);
pan-cytokeratin;
Pro-MMP (pro-matrix-metalloproteinase)-7;
PSA (prostate-specific antigen);
S100A8;
S100A9;
5-100beta;
SCCA1 (squamous cell carcinoma antigen 1);
SCCA2 (squamous cell carcinoma antigen 2);
thyroglobulin;
UHRF1 (ubiquitin-like with/containing PHD and ring-finger domains 1);
URG4 (up-regulated gene 4); and
YKL-40 (synonym: CHI3-L1),
also in combination(s).

10. Method according to claim 1,
characterized in that
heparin or another suitable anticoagulant is used as an agent against coagulation and/or agglomeration.

11. The method according to claim 1,
characterized in that
the perforation or lysis of the macrophages or of the leukocyte populations containing them is performed by means of saponin treatment or a treatment with triton solution.

12. The method according to claim 1,
characterized in that
for determining non blood macrophage antigens, antibodies optionally conjugated with biotin, HRP (horseradish peroxidase), AP (alkaline phosphatase) or luminescent dyes, in particular of the immunoglobulin class (IgG) and Fab or F(ab)₂ fragments and/or aptamers are used, in particular selected from:
anti-mouse IgG (polyclonal);
anti-rabbit IgG (polyclonal);
anti-goat IgG (polyclonal);
anti-rat IgG (polyclonal);
anti-donkey IgG (polyclonal);
anti-AFP (clone AFP-01);
anti-AFP (clone AFP-11);
anti-AFP (clone 4A3);
anti-AFP (clone 5H7);
anti-AFP (clone M803209);
anti-AFP (clone M0151611);
anti-AFP (clone M0151608);
anti-AFP (polyclonal);
anti-AHCY (clone 1E11-1A7);
anti-AHCY (clone 2F11-1D10);
anti-AHCY (clone 4H2);
anti-AHCY (clone MD;
anti-AHCY (clone M2);
anti-AHCY (polyclonal);
anti-AMY2 (clone 6A9/1);
anti-AMY2 (clone 501);
anti-AMY2 (clone 503);
anti-AMY2 (clone 10-102.5);
anti-AMY2 (polyclonal);
anti-CA 15-3/MUC1 (clone M2C5);
anti-CA 15-3/MUC1 (clone M9E7);
anti-CA 15-3/MUC1 (clone M4H2);
anti-CA 15-3/MUC1 (clone M8C9);
anti-CA 15-3/MUC1 (clone M10G4);
anti-CA 15-3/MUC1 (clone M10H6);
anti-CA 15-3/MUC1 (clone M3A106);
anti-CA 15-3/MUC1 (clone C595(NCRC48));
anti-CA 15-3/MUC1 (clone E29);
anti-CA 15-3/MUC1 (polyclonal);
anti-CA 19-9 (clone 121SLE);
anti-CA 19-9 (polyclonal);
anti-CA 50 (clone M991149);
anti-CA 50 (clone 93);
anti-CA 50 (polyclonal);
anti-CA 72-4/TAG72 (clone SPM148);
anti-CA 72-4/TAG72 (polyclonal);
anti-CA 125/MUC16 (clone 2F1);
anti-CA 125/MUC16 (clone 10G12);
anti-CA 125/MUC16 (clone X75);
anti-CA 125/MUC16 (clone X325);
anti-CA 125/MUC16 (polyclonal);
anti-calcitonin (clone SP17);
anti-calcitonin (clone 13B9);
anti-calcitonin (clone 13f2);
anti-calcitonin (clone 24B2);
anti-calcitonin (polyclonal);
anti-calprotectin (clone 27E10);
anti-calprotectin (polyclonal);
anti-CCSA-2 (polyclonal);
anti-CCSP-2 (polyclonal);
anti-CEA (clone Col-1);
anti-CEA (clone 1C7);
anti-CEA (clone 1C10);
anti-CEA (clone 1C11);
anti-CEA (polyclonal);
anti-CYP24A1 (clone 1E1);
anti-CYP24A1 (clone 1F8);
anti-CYP24A1 (polyclonal);
anti-CK8 (clone 24);
anti-CK8 (clone LP3K);
anti-CK8 (polyclonal);
anti-CK18 (clone DC-10);
anti-CK18 (clone DA-7);
anti-CK18 (clone LDK18);
anti-CK18 (polyclonal);
anti-CK19 (clone A53-B/A2);
anti-CK19 (clone BA17);
anti-CK19 (clone 236-11221);
anti-CK19 (polyclonal);
anti-CRP (clone 232007);
anti-CRP (clone 232024);
anti-CRP (clone C2);
anti-CRP (clone C4);
anti-CRP (clone C5);
anti-CRP (clone C6);
anti-CRP (clone C7);
anti-CRP (polyclonal);
anti-cystatin B (clone 2F1);
anti-cystatin B (clone 8k275);
anti-cystatin B (clone B-02);
anti-cystatin B (clone RJMW-2E7);
anti-cystatin B (polyclonal);
anti-DDH (clone T101);
anti-DDH (polyclonal);
anti-DKK-1 (clone 141135);
anti-DKK-1 (polyclonal);
anti-GP73/GOLPH2 (clone YA-14);
anti-GP73/GOLPH2 (clone 5B 10);
anti-GP73/GOLPH2 (polyclonal);
anti-HE4 (clone C-12);
anti-HE4 (polyclonal);
anti-HER2/neu (clone 1007);
anti-HER2/neu (clone 191924);
anti-HER2/neu (clone N3/D 10);
anti-HER2/neu (polyclonal);
anti-HSP-27 (clone G3.1);
anti-HSP-27 (clone AF5E5);
anti-HSP-27 (clone F-4);
anti-HSP-27 (clone 2A5);
anti-HSP-27 (polyclonal);
anti-Mac-2 BP (clone SP-2);
anti-Mac-2 BP (polyclonal);
anti-mammaglobin A (clone 1 G8D6, synonym 2E7G9);
anti-mammaglobin A (clone 304-1A5);
anti-mammaglobin A (polyclonal);
anti-mammaglobin B (clone E-17);
anti-mammaglobin B (polyclonal);
anti-MIA (clone 3A6);
anti-MIA (polyclonal);
anti-MMP-7 (clone 6A4);
anti-MMP-7 (clone 176-5F12);
anti-MMP-7 (clone 141-7B2);
anti-MMP-7 (clone 377313);
anti-MMP-7 (polyclonal);
anti-MnSOD (clone 1AE);
anti-MnSOD (clone 2A1);
anti-MnSOD (clone 4F10);
anti-MnSOD (clone 23G5);
anti-MnSOD (clone 37CT127.5.11.6);
anti-MnSOD (polyclonal);
anti-PARK7/DJ-1 (clone 1B11);
anti-PARK7/DJ-1 (clone 1D7);
anti-PARK7/DJ-1 (clone 6A65);
anti-PARK7/DJ-1 (clone 3055);
anti-PARK7/DJ-1 (clone A-9);
anti-PARK7/DJ-1 (clone D-4);
anti-PARK7/DJ-1 (clone E2);
anti-PARK7/DJ-1 (polyclonal);
anti-proGRP (clone pGRP5);
anti-proGRP (clone E146);
anti-proGRP (clone E172);
anti-GRP (clone 76-E6);
anti-proGRP (polyclonal);
anti-GRP (polyclonal);
anti-NSE (clone 1C1);
anti-NSE (clone 5A4);
anti-NSE (clone 5E2);
anti-NSE (clone 5G10);
anti-NSE (polyclonal);
anti-pan-cytokeratin (clone 7H8C4);
anti-pan-cytokeratin (clone B311.1);
anti-pan-cytokeratin (clone C11);
anti-pan-cytokeratin (clone D-12);
anti-pan-cytokeratin (polyclonal);
anti-PSA (clone ER-PR8);
anti-PSA (clone 181823);
anti-PSA (polyclonal);
anti-S100A8 (clone 1B3);
anti-S 100A8 (clone 2C5/4);
anti-S 100A8 (clone 2H2);
anti-S100A8 (clone 2Q396A);
anti-S100A8 (clone 6A614);
anti-S100A8 (clone 8L627);
anti-S100A8 (clone 8-5C2);
anti-S100A8 (clone CF-145);
anti-S100A8 (clone MRP8 7C12/4);
anti-S100A8 (clone S13.67);
anti-S 100A8 (polyclonal);
anti-S100A9 (clone 1C10);
anti-S100A9 (clone 2Q396B);
anti-S100A9 (clone 4G9);
anti-S100A9 (clone NO.19);
anti-S100A9 (clone NO.134);
anti-S100A9 (clone S32.2);
anti-S100A9 (clone S36.48);
anti-S 100A9 (polyclonal);
anti-S-100beta (clone SB6);
anti-S-100beta (clone SH-B1);
anti-S-l00beta (clone SH-B4);
anti-S-100beta (polyclonal);
anti-SCCA1 (clone 8H11);
anti-SCCA1 (polyclonal);
anti-SCCA2 (clone 10C12);
anti-SCCA2 (polyclonal);
anti-SCCA1/2 (clone B-9);
anti-SCCA1/2 (polyclonal);
anti-thyroglobulin (clone 5E6);
anti-thyroglobulin (clone 5F9);
anti-thyroglobulin (clone 5G4);
anti-thyroglobulin (clone 11A16);
anti-thyroglobulin (clone PB2);
anti-thyroglobulin (clone PB3);
anti-thyroglobulin (polyclonal);
anti-UHRF1 (clone 1RC1C-10);
anti-UHRF1 (clone 3A11);
anti-UHRF1 (polyclonal);
anti-URG4 (polyclonal);
anti-YKL-40/CHI3-L1 (clone 2011);
anti-YKL-40/CHI3-L1 (clone 321806);
anti-YKL-40/CHI3-L1 (polyclonal).

13. An assembly for isolating and characterizing mononuclear leukocytes of the blood, in particular blood macrophages, including:
a device for adding an agent inhibiting coagulation and/or agglomeration of whole blood;
a device for performing a pre-selection and enrichment of mononuclear leukocytes, in particular blood macrophages, from whole blood by means of density gradient centrifugation;
a device for performing a selection and enrichment of CD14⁺ cells by antibodies against CD14, which are, optionally reversibly, coupled to magnetic beads or to an Elisa plate;
a device for performing a selection and enrichment of CD16⁺ cells by antibodies against CD16, which are, optionally reversibly, coupled to magnetic beads or to an Elisa plate;
a device for perforation or lysis of the mononuclear leukocyte populations, in particular blood macrophages, by means of a saponin or triton solution;
a device for quantitative determination of the mononuclear leukocyte populations, in particular blood macrophages, using an assay for determining the activity of the LDH (lactate dehydrogenase) in an Elisa plate, in the same Elisa plate, in which the qualitative and quantitative determination of the molecular markers is performed;
a device for qualitative and quantitative determination of intracellular molecular markers phagocytosed by blood macrophages after preceding perforation of lysis of the mononuclear leukocytes, in particular macrophages, of the blood by means of Elisa plate reader and/or a chemiluminescence measuring device.

14. The assembly according to claim 13,
characterized by
a device for excluding NK cells (natural killer cells) with the cell surface markers CD56 or CD57 or CD161 from the collective of mononuclear leukocytes, in particular CD16⁺ leukocytes, using antibodies against CD56 or CD57 or CD161, which are coupled to magnetic beads.

15. The assembly according to claim 13,
characterized by
a device for fixation and permeabilization of mononuclear leukocytes before lysis thereof, preferably before or optionally after the selection and/or enrichment and/or separation of the blood macrophages or leukocyte populations containing blood macrophages for intracellular binding of antibodies directed against intracellular phagocytosed molecular markers before cell lysis.

16. The method according to claim 2,
characterized in that
a negative selection for excluding cells with the surface markers CD56 and/or CD57 and/or CD161 is performed using antibodies directed against the surface markers CD56 and/or CD57 and/or CD161.

17. The method according to claim 3,
characterized in that
a negative selection for excluding cells with the surface markers CD56 and/or CD57 and/or CD161 is performed using antibodies directed against the surface markers CD56 and/or CD57 and/or CD161.

18. The method according to claim 2,
characterized in that
a positive or negative selection is performed using magnetic beads, which can be, preferably reversibly, coupled.

19. The method according to claim 3,
characterized in that
a positive or negative selection is performed using magnetic beads, which can be, preferably reversibly, coupled.

20. The method according to claim 4,
characterized in that
a positive or negative selection is performed using magnetic beads, which can be, preferably reversibly, coupled.
